(19) United States
(12) Patent Application Publication
(10) Pub. No.: US 2022/0017933 A1
(43) Pub. Date: Jan. 20, 2022

(54) METHOD FOR THE PRODUCTION OF ENZYMES BY A STRAIN BELONGING TO A FILAMENTOUS FUNGUS

(71) Applicant: IFP Energies nouvelles, Rueil-Malmaison (FR)

(72) Inventors: Fadhel BEN CHAABANE, Rueil-Malmaison (FR); Celine COHEN, Rueil-Malmaison (FR)

(73) Assignee: IFP Energies nouvelles, Rueil-Malmaison (FR)

(21) Appl. No.: 17/295,198

(22) PCT Filed: Nov. 4, 2019
(86) PCT No.: PCT/EP2019/080130

§ 371 Date: May 19, 2021

(30) Foreign Application Priority Data
Nov. 26, 2018 (FR) FR1871839

(51) Int. Cl. C12P 21/02; C12N 1/14; C12N 1/22; C12N 9/42

(52) U.S. Cl. CPC C12P 21/02; C12N 1/14; C12R 2001/885; C12N 9/2437; C12N 1/22

(57) ABSTRACT

The present invention concerns a process for producing enzymes by a strain belonging to a filamentous fungus, said process comprising two steps:
(a) a first step of growing the fungi, in the presence of at least one carbon-based growth substrate, in a stirred and aerated bioreactor in batch phase, at a pH of not more than 4.6;
(b) a second step of producing enzymes, starting from the culture medium obtained in the first step (a), in the presence of at least one inductive carbon-based substrate, at a pH of not more than 4.6.

### TECHNICAL FIELD

The invention relates to a process for producing cellulases with a filamentous fungus, required for the enzymatic hydrolysis of lignocellulosic biomass used, for example, in processes for producing “second generation” (2G) sugary liquors. These sugary liquors may be used to produce other products via a chemical or biochemical/fermentation pathway (for example alcohols such as ethanol biofuels, or else butanol or other molecules, for example solvents such as acetone and other biobased molecules, etc.). Cellulases may also be used in other processes, notably in the chemical, paper or textile industry.

The development of economically viable processes for producing second-generation (2G) biofuels, to take this particular example of implementation, is the subject of numerous studies. These biofuels are notably produced from ligneous substrates such as various woods (hardwood and softwood, miscanthus, or SRC, which is the abbreviation for Short-Rotation Coppice), agricultural byproducts (wheat straw, rice straw, corn cobs, etc.) or byproducts from other agrifood, paper, etc. industries. They pose fewer problems of competition with subsistence crops for the use of agricultural land, when compared with “first-generation” biofuels which are produced from sugarcane, corn, wheat or beet.

Lignocellulosic biomass is characterized by a complex structure composed of three main fractions: cellulose (35% to 50%), which is a polysaccharide essentially constituted of hexoses; hemicellulose (20% to 30%), which is a polysaccharide essentially constituted of pentoses; and lignin (15% to 25%), which is a polymer of complex structure and of high molecular weight, composed of aromatic alcohols connected via ether bonds. These various molecules are responsible for the intrinsic properties of the plant wall and are organized in a complex entanglement. Among the three base polymers that make up lignocellulosic biomass, cellulose and hemicellulose are the ones that enable the production of 2G sugary liquors.

Conventionally, the process for transforming biomass into ethanol biofuel involves several steps: Pretreatment makes the cellulose accessible to the cellulase enzymes. The enzymatic hydrolysis step allows the transformation of cellulose into sugars, such as glucose, which are then transformed into ethanol during the fermentation step, generally with the yeast Saccharomyces cerevisiae. Finally, the distillation step makes it possible to separate and recover the ethanol from the fermentation must.

It should be noted, as mentioned above, that another possible choice is to stop the process at the production of glucose-type sugars, in order to utilize them as such, or else to process them differently in order to obtain other biobased alcohols or molecules.

### PRIOR ART

Various technico-economic studies demonstrate that reducing the cost of cellulases is one of the key points in processes for the biological production of ethanol from lignocellulosic raw materials. At the present time, industrial cellulases are mainly produced by a filamentous fungus, Trichoderma reesei, on account of its high secretory power.

Since the 1970s, the transformation of lignocellulosic materials into ethanol, after hydrolysis of the constituent polysaccharides into fermentable sugars, has been the subject of numerous studies. Mention may be made, for example, of the reference studies by the National Renewable Energy Laboratory (Process Design and Economics for Biochemical Conversion of Lignocellulosic Biomass to Ethanol, Humbird et al., NREL/TP-5100-57764, May 2011).

Lignocellulosic materials are cellulose-based materials, i.e. materials consisting to more than 90% by weight of cellulose, and/or are lignocellulosic materials, i.e. materials consisting of cellulose, hemicelluloses, which are polysaccharides essentially consisting of pentoses and hexoses, and also lignin, which is a macromolecule of complex structure and of high molecular weight, based on phenolic compounds.

Wood, straw and corn cobs are the lignocellulosic materials most commonly used, but other resources, dedicated forestry crops, residues from alcohol-yielding, sugar-yielding and cereal plants, products and residues from the paper industry and products from the transformation of lignocellulosic materials are usable. They are for the majority constituted of about 35% to 50% of cellulose, 20% to 30% of hemicellulose and 15% to 25% of lignin.

The process of biochemical transformation of lignocellulosic materials into ethanol comprises a physicochemical pretreatment step, followed by a step of enzymatic hydrolysis using an enzyme cocktail, a step of ethanolic fermentation of the sugars released, the ethanolic fermentation and the enzymatic hydrolysis possibly being conducted simultaneously, and a step of purification of the ethanol.

The enzyme cocktail is a mixture of cellulolytic enzymes (also known as cellulases) and/or hemicellulolytic enzymes. Cellulolytic enzymes have three major types of activities: endoglucanases, exoglucanases and cellobiases, the latter also being known as β-glucosidases. Hemicellulolytic enzymes notably have xylanase activities.

Enzymatic hydrolysis is efficient and is performed under mild conditions. However, the cost of the enzymes remains very high, representing from 20% to 50% of the cost of transformation of lignocellulosic material into ethanol. As a result, numerous studies have been conducted to reduce this cost: first, optimization of the production of enzymes, by selecting hyper-productive microorganisms and by improving the processes for producing said enzymes, reduction of the amount of enzymes subsequently in hydrolysis, by optimizing the pretreatment step, by improving the specific activity of these enzymes, and by optimizing the implementation of the enzymatic hydrolysis step.

Numerous studies have focused on understanding the mechanisms of action and expression of the enzyme cocktail. The aim is to secrete the cocktail that is the most suitable for the hydrolysis of the lignocellulosic materials by modifying the microorganisms.

Trichoderma reesei is the microorganism most widely used for the production of cellulases. The wild-type strains have the faculty of excreting, in the presence of an inductive substrate, for example cellulose, the enzymatic complex considered as being the best suited for the hydrolysis of cellulose. The enzymes of the enzymatic complex contain three main types of activities: endoglucanases, exoglucanases and cellobiases and other proteins which have properties that are essential for the hydrolysis of lignocellulosic materials are also produced by Trichoderma reesei, for example xylanases. The presence of an inductive substrate is essential for the expression of the cellulolytic and/or hemicellulolytic enzymes. The nature of the carbon-based substrate has a strong influence on the composition of the enzymatic complex. This is the case for xyloses, which, when combined with a carbon-based inductive substrate such as cellulose or lactose, make it possible to significantly improve the activity of said xylanase. Regulation of the cellulase genes has been studied in detail on a variety of carbon sources. They are induced in the presence of cellulose, of its hydrolysis products, such as cellobiose, or of certain oligosaccharides such as lactose or sophorose (cf. Ilmén et al., 1997; Appl. Environ. Microbiol. 63: 1298-1306).

Conventional genetic mutation techniques have enabled the selection of strains of Trichoderma reesei which hyperproduce cellulases, such as the strains MCG77 (Gallo—patent U.S. Pat. No. 4,275,167), MCG 80 (Allen, A. L. and Andreotti, R. E., Biotechnol.-Bioeng. 1982, 12, 451-459 1982), RUT C30 (Montenecourt, B. S. and Eveleigh, D. E., Appl. Environ. Microbiol. 1977, 34, 777-782) and CL847 (Durand et al., 1984, Proc. Colloque SFM “Génétique des microorganismes industriels [Genetics of industrial microorganisms]”. Paris. H. Heslot Ed, pages 39-50).

The process for producing cellulases by Trichoderma reesei has been the subject of substantial improvements for the purpose of extrapolation to the industrial scale. To obtain good enzyme productivities, it is necessary to supply a source of rapidly assimilable carbon for the growth of Trichoderma reesei and an inductive substrate which allows the expression of the cellulases and secretion into the culture medium. Cellulose can play these two roles; however, it is difficult to use at the industrial stage and it has been proposed to replace it with soluble carbon sources, such as glucose, xylose or lactose, lactose also acting as inductive substrate. Other soluble sugars such as cellobiose and sophorose have been described as inductive, but they are relatively expensive for use at the industrial stage. It has also been found that productions of cellulases by Trichoderma reesei, with soluble substrates, are very much inferior to those obtained on cellulose by “batch”. This is due to the repressor effect of the readily assimilable sugars, at high concentration. Continuous feeding in fed-batch mode of soluble carbon-based substrates has made it possible to raise the catabolic repression by limiting the residual concentration in the cultures and by optimizing the amount of sugar, making it possible to obtain a better yield and better enzymatic productivity.

Patent FR-B-2 555 603 proposes a protocol for arriving at a protein concentration of the order of 35 to 40 g/L with a productivity of the order of 0.2 g/Uh, which consists of two steps: a first step of growth in “batch” mode in which it is necessary to supply a source of rapidly assimilable carbon for the growth of Trichoderma reesei, and then a step of production in “fed-batch” mode using an inductive substrate (for example: lactose) which allows the expression of the cellulases and secretion into the culture medium. The optimum flow applied is between 35 and 45 mg·g⁻¹·h⁻¹ (milligrams of inductive substrate per milligram of biomass per hour). Mention may also be made of patent EP-B-2 744 899, which proposes an improvement thereto, by notably selecting a bioreactor which has a particular coefficient of volumetric transfer of oxygen, KLa, combined with a particular selection both of the concentration of carbon-based growth substrate in the first step and of a level of flow limiting the carbon source in the second step.

It has nevertheless emerged that a foam may be formed, more particularly during the growth step. This may be a “dry” foam, meaning one composed of a dispersion of gas in a liquid phase, whose density is therefore close to that of a gas and which forms in the upper part of the bioreactor. It may also be a “wet” foam, this being a foam that extends/increases the reaction volume through the trapping of gas (air) bubbles in the liquid. It has a greater density than the dry foam (by virtue of the lower gas/liquid ratio). Whether one or the other or a mixture of both of these foam types, it presents a real problem to industrial implementation of the process. The reason is that the presence of the foams, to state only some of their disbenefits, seriously complicates the pH regulation usually performed during the growth step, as pH measurement becomes harder/less reliable with foam present, and also as it is more complicated to add pH regulator to maintain the desired pH, it being more difficult to control the distribution of the regulator throughout the reaction medium. The bioreactors also have to be utilized at reduced capacity, to leave sufficient space above the liquid reaction medium, to prevent any overflow. A number of solutions have already been proposed to combat foam forming. A first solution was to add antifoams to the reaction medium during the growth step. While using antifoams is certainly effective at resuspending the foam as a liquid, it is not without disbenefits. To state a few of them: resuspending the foam as a liquid gives rise to a large increase in the pH, greatly disrupting the necessary regulation of pH, and even provoking an unwanted tipover from the growth step into the production step. This may be caused by the mass supply of reactants (sugars) blocked at the surface owing to the foam, which, as it breaks down under the effect of the antifoam, come abruptly into contact with the biomass in large quantity. The addition of antifoams also causes a drop in the concentration of dissolved oxygen in the medium (since it causes the air bubbles to coalesce), and this may have an impact on the microorganisms which are obligate aerobes or on their productivity. These antifoams, moreover, are often oils, which are not eliminated by themselves: when enzyme production is at an end, if the enzymes are separated from the rest of the biomass (the fungi), especially by conventional technologies using membrane filtration means, these antifoams can cause plugging of the membranes, and it may therefore be necessary to add a step for separating these antifoams when production is finished; if not, separation performance is poor. Adding them is also an additional production cost.

Patent application EP 1 204 738 considered a different solution: it involves combating this foaming phenomenon by genetically modifying the strain of fungus used, so as to prevent the strain secreting hydrophobins, and especially the HFBIIs, which are held responsible for the formation of foam. However, this solution is laborious to implement, since it requires that these genetic modifications be performed on each of the strains of interest.

The aim of the invention is thus to develop an improved enzyme production process that avoids or at least limits the phenomenon of foaming, without giving rise to some at least of the abovementioned disbenefits, and in particular without complicating the implementation of the process or requiring specific genetic modifications to be performed on the microorganisms.

### SUMMARY OF THE INVENTION

The invention first provides a process for producing enzymes by a strain belonging to a filamentous fungus, said process comprising two steps:

(a) a first step of growing the fungi, in the presence of at least one carbon-based growth substrate, in a stirred and aerated bioreactor in batch phase, at a pH of not more than 4.6;

(b) a second step of producing enzymes, starting from the culture medium obtained in the first step (a), in the presence of at least one inductive carbon-based substrate, at a pH of not more than 4.6.

The choice made according to the invention, then, is to carry out not only the second, enzyme production step at a relatively acidic pH, but also the first, growth step, hitherto carried out at less acidic pH levels, of at least 5, for example. Whereas operating the first step at such an acidic pH would have been expected to result in a slowing of microorganism growth, it emerged that no such effect occurred, and also that, wholly surprisingly, the incidence of foam during this step was prevented or greatly limited. By lowering the pH accordingly during the growth step, control is exerted over the foaming problem without impacting the eventual production yield of enzymes.

The pH of the growth step is preferably regulated to hold it within the required range. The pH of the production step is preferably regulated too. Regulation is accomplished conventionally, in particular by continuous or sequential pH monitoring with ad hoc sensors, and addition of acid or base during the step to stay within the confines. The pH of one and/or the other of the steps may alternatively be controlled using a buffer solution.

The solution of the invention is astoundingly simple, since there was nothing to predict that modifying the pH, in the direction of greater acidity, within reasonable proportions (preferably without going below 3.5 or 3.6 or 3.7), during the growth phase would affect the complex phenomenon of foaming. It is highly advantageous in terms of implementation of industrial production:—the bioreactor in which the growth step is carried out is comprehensively equipped to regulate the pH at these values, and so it is not difficult at all to perform the invention with conventional bioreactors;—since very little or no foam is formed, the size of the bioreactor can be calculated exactly, and its useful volume increased (there is no longer any need to provide for an extra “lost” volume to contain any excessive overflows of foam);
it is no longer necessary to add antifoams, or at the very least the amount thereof can be reduced very greatly, so avoiding an additive which is subsequently difficult to separate;
it is easier to control the bioreactor, since, in the absence of foam, the regulation of pH, the controlled addition of carbon-based substrate, etc., are all greatly facilitated.

The pH in the growth step (a) and/or in the production step (b) is preferably at least 3.5, and in particular not more than 4.4, being in particular between 3.5 and 4.4 or between 3.8 and 4.4: in this way the pH of the growth step is brought closer to the pH of the production step.

The growth step pH is preferably maintained at not less than 3.6, in particular at least 3.7 or at least 3.8.

The growth step pH is preferably maintained at not more than 4.4.

In one embodiment, the pH in the growth step (a) is substantially identical to the pH in the production step (b). If, in particular, the two steps are conducted in the same bioreactor, selecting the same pH values thus simplifies pH regulation throughout the process duration. It is possible, then, to have the same regulation setpoint over both steps or to use the same buffer solution.

In another embodiment, the pH in the production step (b) may be selected to be more acidic than the pH in the growth step (a), by at least 0.3 to 0.6, for example, in particular a pH which is more acidic (and therefore lower) by 0.4 to 0.6.

Advantageously, the pH is regulated during the growth step (a) by controlled addition of a nitrogen compound, especially aqueous ammonia, which acts both as a basic agent and as a source of nitrogen for the growth of the microorganisms.

The production step (b) operates advantageously in batch, fed-batch or continuous mode or in two or more of these modes successively.

Optionally, the process according to the invention may comprise an intermediate step (c) between step (a) and step (b), this intermediate step (c) being a step of diluting the culture medium obtained in the growth step (a).

In addition, the growth step (a) and the production step (b) may be performed in the same bioreactor or in two different reactors, with transfer of the reaction medium from one reactor to the other. The first case is the simpler: with only one reactor, the need to transfer the reaction medium is avoided. The second case enables precise adaptation of the characteristics and equipment of each of the bioreactors as a function of the needs of each of the steps.

Preferably, during the first, growth step (a), the selected concentration of carbon-based growth substrate is between 15 and 60 g/L.

The second, production step (b) is preferably operated with a limiting stream of inductive carbon-based substrate, notably of between 30 and 140 mg·g⁻¹·h⁻¹ (that is, between 30 and 140 grams per gram of biomass per hour), preferably between 35 and 45 mg·g⁻¹·h⁻¹, and preferably with an aqueous solution of inductive carbon-based substrate at a concentration of between 200 and 600 g/L.

The strain used in the process according to the invention is preferably a strain of Trichoderma reesei or of Trichoderma reesei modified by selective mutation or genetic recombination. It is, though, not useful to modify the strain genetically with the aim of preventing it forming hydrophobins as it grows. The strain may notably be a strain CL847, RutC30, MCG77 or MCG80 as mentioned earlier on above.

The process according to the invention preferably produces cellulolytic and/or hemicellulolytic enzymes (cellulases).

The process according to the invention is advantageously operated in the absence of antifoams, in particular during the production step (a). No longer employing antifoams is highly advantageous economically. Furthermore, the addition of these antifoams can cause problems involving limitation of oxygen transfer from the air supplied to the bioreactor to the liquid phase comprising the fungi, which is detrimental to their growth. These antifoams may additionally pose problems when the culture medium is filtered at the end of the production step.

The invention also provides for the use of the enzymes obtained by the process described above for the enzymatic hydrolysis of terrestrial or marine cellulosic/hemicellulosic biomass.

The invention will be described below in greater detail with the aid of non-limiting working examples.

### LIST OF FIGURES

FIG. 1 shows photos of the bioreactors used in examples compliant and not compliant with the process of the invention with one strain, the photos taken during the growth step of the process.

FIG. 2 shows photos of the bioreactors used in examples compliant and not compliant with the process of the invention with a different strain, the photos taken during the growth step of the process.

FIG. 3 is a graph showing the concentration in grams per liter of biomass and proteins produced in a comparative example.

FIG. 4 is a graph showing the concentration in grams per liter of biomass and proteins produced in a working example of the invention.

FIG. 5 is a graph showing the concentration in grams per liter of biomass and proteins produced in a working example of the invention.

### DESCRIPTION OF THE EMBODIMENTS

The present invention has developed a process for producing enzymes, especially cellulases, in which the incidence of foam is prevented. It has surprisingly emerged that operating at low pH levels during growth (of lower than 4.6, in particular not more than 4.4) does not slow growth of the microorganism and prevents the incidence of foam.

The process regime comprises 2 phases:
a batch mode phase lasting preferably between 30 and 50 hours, with a pH advantageously of not more than 4.4 and with a concentration of carbon-based substrate of 15 to 60 g/L preferably
a fed-batch mode phase lasting preferably between 100 and 200 hours, in particular between 100 and 150 hours, advantageously at a pH likewise of not more than 4.4, and with a limiting stream of carbon source of from 35 to 140 mg·g⁻¹·h⁻¹ and preferably between 35 and 45 mg·g⁻¹·h⁻¹.

The industrial strains used belong to the species Trichoderma reesei, and are modified to enhance the cellulolytic and/or hemicellulolytic enzymes by mutation-selection methods, an example being the strain CL847 (one such method is described in particular in U.S. Pat. No. 4,762,788). Strains enhanced by genetic recombination techniques may also be used. These strains are cultured in stirred and aerated fermenters under conditions compatible with their growth and the production of the enzymes.

The main carbon sources may be soluble sugars such as lactose, glucose or xylose:

The carbon-based growth substrate is preferably selected from lactose, glucose, xylose, residues obtained after ethanolic fermentation of monomeric sugars from the enzymatic hydrolysates of cellulosic biomass, and/or a crude extract of water-soluble pentoses from the pretreatment of a cellulosic biomass.

The inductive carbon-based substrate is preferably selected from lactose, cellobiose, sophorose, residues obtained after ethanolic fermentation of monomeric sugars from the enzymatic hydrolysates of cellulosic biomass, and/or a crude extract of water-soluble pentoses from the pretreatment of a cellulosic biomass.

This type of residue/extract may thus also be used as a total carbon source, i.e. both for the growth of the microorganism and for the induction of the expression system. This carbon source can be utilized more particularly by genetically enhanced strains and, especially, recombinant strains.

The operating conditions of pH and temperature, for the growth step and the production step, are as follows:
pH between 3.5 and 4.4;
temperature between 20 and 35° C. Preference is given to selecting a pH of 4.4 and a temperature of 27° C. during the growth phase, and a pH of 4 or of likewise 4.4 and a temperature of 25° C. during the fed-batch mode production phase.

The vvm (degree of aeration expressed as volume of air per unit volume of reaction medium per minute) applied during the process is between 0.3 and 1.5 min⁻¹ and the rpm (stirring speed) must allow the O₂ pressure to be regulated to between 20% and 60%. An aeration of 0.3 to 0.5 vvm and stirring which allows the O₂ pressure to be regulated to 30% or 40% are preferably selected.

Depending on its nature, the carbon-based substrate selected for producing the biomass is introduced into the fermenter before sterilization, or is sterilized separately and introduced into the fermenter after sterilization. The concentration of carbon-based substrate is between 200 and 600 g/L depending on the degree of solubility of the carbon-based substrates used (notably as regards the inductive substrate).

### EXAMPLES

The strains are precultured in 2 Fernbach flasks with a useful capacity of 500 mL, which are seeded with one tube each of T. reesei TR3002 and CL847 spores. They are placed in an INFORS HT Multitron incubator at 30° C., with orbital stirring at 150 rpm for 72 hours. They are then consigned to 8 vacuum-sterilized flasks (80 mL per flask) which will be used to seed each fermenter/bioreactors.

The operating conditions for producing cellulases from the strains obtained after preculturing are as follows:

The experiments comprise two phases:
a growth phase on glucose at a temperature of 27° C. and a pH (regulated using 5.5 M aqueous ammonia) ranging from 4.0 to 5.5 according to experiment. Aeration is set at 0.2 L/min (0.3 vvm) and the setpoint pO₂ at 40%. Kept constant by virtue of the stirring is
an enzyme production phase induced by a limiting stream of the fed-batch solution after 30 hours' culturing. The solution is injected with a constant rate of 0.5 g of carbon-based substrate per hour. The setpoint temperature is modified to 25° C. This phase lasts between 160 and 220 hours according to substrate availability and experimental progression.

Sampling takes place each day, with monitoring of the dry weight and the concentrations of glucose, lactose, galactose, and xylose. 5 mL culture supernatants are stored at 4° C. for protein and enzyme assays conducted at the end of culturing.

### Example 1

Example 1 is carried out starting from the strain TR3002. This strain is described in the following publications: Ben Chaabane F, Jourdier E, Licht R, Cohen C and Monot F (2012) “Kinetic characterization of Trichoderma reesei CL847 TR3002: an engineered strain producing highly improved cellulolytic cocktail”, Journal of Chemistry and Chemical Engineering 6 (2), 109-117, and Ayrinhac C, Margeot A, Ferreira N L, Ben Chaabane F, Monot F, Ravot G, Sonet J.-M and Fourage L (2011) “Improved saccharification of wheat straw for biofuel production using an engineered secretome of Trichoderma reesei”, Organic Process Research and Development 15 (1), 275-278.
The growth phase is conducted at pH 4, at 27° C. and with a glucose concentration of 15 g/L
The production phase is conducted at pH 4, at 25° C. and with a lactose concentration of 220 g/L, corresponding to a specific fed-batch lactose flow rate of 45 mg per gram of biomass per hour.

### Example 2: (Comparative)

Example 2 is carried out starting from the strain TR3002.
The growth phase is conducted at pH 4.8, at 27° C. and with a glucose concentration of 15 g/L
The production phase is conducted at pH 4.8, at 25° C. and with a lactose concentration of 220 g/L, corresponding to a specific fed-batch lactose flow rate of 45 mg per gram of biomass per hour.

### Example 3: (Comparative)

Example 3 is carried out starting from the strain TR3002.
The growth phase is conducted at pH 5.5, at 27° C. and with a glucose concentration of 15 g/L
The production phase is conducted at pH 5.5, at 25° C. and with a lactose concentration of 220 g/L, corresponding to a specific fed-batch lactose flow rate of 45 mg per gram of biomass per hour.

### Example 4

Example 4 is carried out starting from the strain TR3002.
The growth phase is conducted at pH 4.4, at 27° C. and with a glucose concentration of 15 g/L
The production phase is conducted at pH 4.4, at 25° C. and with a lactose concentration of 220 g/L, corresponding to a specific fed-batch lactose flow rate of 45 mg per gram of biomass per hour.

### Example 5

Example 5 is carried out starting from the strain CL847. This strain is described in the following publications:—Jourdier E, Poughon L, Larroche C, Monot F and Ben Chaabane F (2012) “A new stoichiometric miniaturization strategy for screening of industrial microbial strains: application to cellulase hyper-producing Trichoderma reesei strains”, Microbial Cell Factories 11, 70 (Impact Factor: 3,60), and Jourdier E, Ben Chaabane F, Poughon L, Larroche C and Monot F (2012) “Simple Kinetic Model of Cellulase Production by Trichoderma Reesei for Productivity or Yield Maximization”, Chemical Engineering Science 27, 313-318.
The growth phase is conducted at pH 4, at 27° C. and with a glucose concentration of 15 g/L
The production phase is conducted at pH 4, at 25° C. and with a lactose concentration of 220 g/L, corresponding to a specific fed-batch lactose flow rate of 45 mg per gram of biomass per hour.

### Example 6

Example 6 is carried out starting from the strain CL847.
The growth phase is conducted at pH 4.4, at 27° C. and with a glucose concentration of 15 g/L
The production phase is conducted at pH 4.4, at 25° C. and with a lactose concentration of 220 g/L, corresponding to a specific fed-batch lactose flow rate of 45 mg per gram of biomass per hour.

### Example 7: (Comparative)

Example 7 is carried out starting from the strain CL847.
The growth phase is conducted at pH 4.8, at 27° C. and with a glucose concentration of 15 g/L
The production phase is conducted at pH 4.8, at 25° C. and with a lactose concentration of 220 g/L, corresponding to a specific fed-batch lactose flow rate of 45 mg per gram of biomass per hour.

### Example 8: (Comparative)

Example 8 is carried out starting from the strain CL847.
The growth phase is conducted at pH 5.5, at 27° C. and with a glucose concentration of 15 g/L
The production phase is conducted at pH 5.5, at 25° C. and with a lactose concentration of 220 g/L, corresponding to a specific fed-batch lactose flow rate of 45 mg per gram of biomass per hour.

Visual observation of the incidence or nonincidence of foam leads to the results collated in table 1 below:

TABLE 1

| Example | Strain | Growth pH | Visual observation |
|---|---|---|---|
| Example 1 | TR3002 | 4 | No foam |
| Example 2 | TR3002 | 4.8 | Foam from 24 h then continuously |
| Example 3 | TR3002 | 5.5 | Foam from 24 h then continuously |
| Example 4 | TR3002 | 4.4 | No foam |
| Example 5 | CL847 | 4 | No foam |
| Example 6 | CL847 | 4.4 | No foam |
| Example 7 | CL847 | 4.8 | Foam from 24 h then continuously |
| | | | until sporulation |
| Example 8 | CL847 | 5.5 | Foam from 24 h then continuously |
| | | | until sporulation |

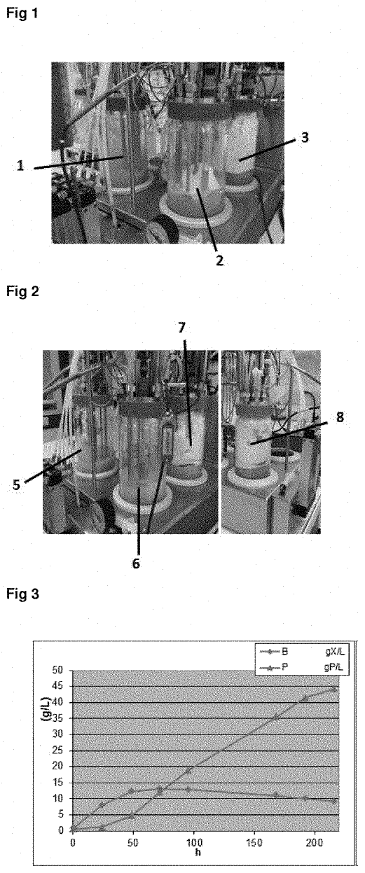

FIGS. 1 and 2 show photographs of the bioreactors of examples 1, 2, 3, 5, 6, 7 and 8 after 24 hours: (reference 1 corresponds to example 1 and so on).

From table 1 and FIGS. 1 and 2 it is noted that whichever strain is used (CL847 or TR3002), a growth-phase pH greater than or equal to 4.8 results in production of foam from 24 hours onward. After that, this foam cannot be controlled, and so the fermentations stop at a fairly high pH owing to overflow or sporulation. Conversely there was no foaming with the cultures at a pH of not more than 4.4.

To ascertain whether the selection of the growth pH at values of not more than 4.4 in accordance with the invention had any effect otherwise on the performance of the strain, a calculation was made of the specific protein production rate qp. This specific rate qp is equal to rp/X, where rp is the protein productivity in g/L/h, and X is the concentration of biomass in g/L. The values obtained for qp are as follows:

Example 1 (pH 4 growth, strain TR3002): qp=28.4 mgP/gX/h

Example 2 (pH 4.8 growth, strain TR3002): qp=27.7 mgP/gX/h

Example 4 (pH 4.4 growth, strain TR3002): qp=25.4 mgP/gX/h

Example 5 (pH 4 growth, strain CL847): qp=8.5 mgP/gX/h

Example 6 (pH 4.4 growth, strain CL847): qp=9.4 mgP/gX/h

For examples 7 and 8, the values of qp are not significantly different from those of examples 5 and 6 while no foaming has started, but foaming thereafter interrupted the experiments for these two examples.

It is therefore found that, for a given strain, lowering the growth pH had no significant effect on either the specific rate qp or the overall productivity of the process. Indeed, the final concentration of proteins, for a given production time, is no longer affected by the lowering of the growth pH: amounts of around 40 g/L are obtained for the examples with strain TR3002 irrespective of growth pH, and amounts of around 25 g/L for the examples with strain CL847 irrespective of growth pH.

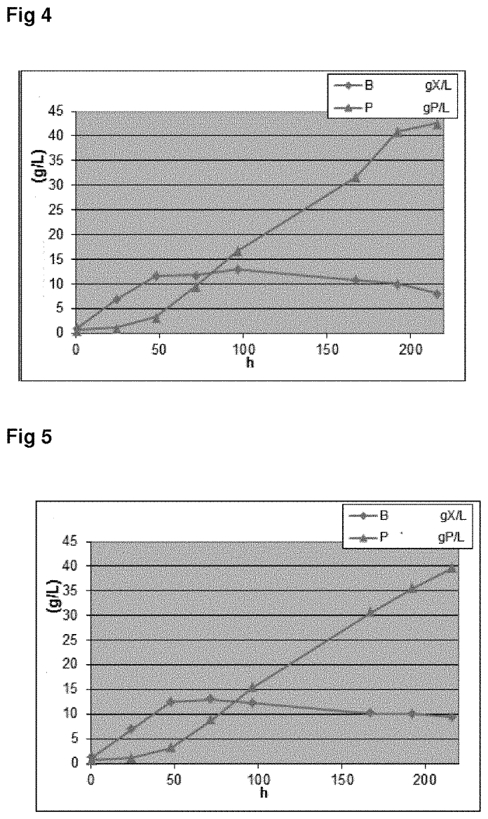

FIG. 3 indicates the production in g/L of biomass (line with dots) and of proteins (line with triangles) as a function of time expressed in hours for comparative example 2, with FIG. 4 showing the same type of graph for inventive example 1, and FIG. 5 for inventive example 4: comparison of these graphs confirms that protein production remains at the same level whether or not the growth pH is lowered. It is also apparent (example 4, FIG. 5) that adopting the same pH for growth and production no longer has any adverse effect on protein production. Additionally the enzymatic activity levels of the enzymes produced were evaluated, by measurement of what is called the Filter Paper Activity (“FPase”). This method allows assaying of the overall activity of the enzymic pool (endoglucanases and exoglucanases). The FPase activity is measured on Whatman #1 paper (procedure recommended by the IUPAC biotechnology commission): A determination is made of the sample of the enzymatic solution that produces a 4% advancement of the enzymatic reaction in 60 minutes. The principle of the filter paper activity is to use a DNS (dinitrosalicylic acid) assay to determine the amount of reduced sugars obtained from a Whatman #1 paper.

The FPase values obtained for the 8 examples, which thus convey the overall activity of the enzyme cocktail, and hence its quality, exhibit values which are conventional for the strains used, of between 0.8 and 1 IU/mg.

The conclusion drawn from this is that although the invention enables particularly simple and effective prevention of foaming, it has no negative impact on either the enzyme production yield or the quality of the enzymes produced.

1. A process for producing enzymes by a strain belonging to a filamentous fungus, characterized in that said process comprises two steps:
(a) a first step of growing the fungi, in the presence of at least one carbon-based growth substrate, in a stirred and aerated bioreactor in batch phase, at a pH of not more than 4.6;
(b) a second step of producing enzymes, starting from the culture medium obtained in the first step (a), in the presence of at least one inductive carbon-based substrate, at a pH of not more than 4.6.

2. The process as claimed in claim 1, characterized in that the pH in the growth step (a) and/or in the production step (b) is not more than 4.4, in particular between 3.5 and 4.4.

3. The process as claimed in claim 1, characterized in that the pH in the growth step (a) is substantially identical to the pH in the production step (b).

4. The process as claimed in claim 1, characterized in that the pH in the production step (b) is more acidic than the pH in the growth step (a).

5. The process as claimed in claim 1, characterized in that the pH is regulated during the growth step (a) by controlled addition of a nitrogen compound, especially aqueous ammonia.

6. The process as claimed in claim 1, characterized in that the production step (b) operates in batch, fed-batch or continuous mode, or in two or more of these modes successively.

7. The process as claimed in claim 1, characterized in that it comprises an intermediate step (c) between step (a) and step (b), this intermediate step (c) being a step of diluting the culture medium obtained in the growth step (a).

8. The process as claimed in claim 1, characterized in that the concentration of carbon-based growth substrate during the first, growth step (a) is between 15 and 60 g/L.

9. The process as claimed in claim 1, characterized in that the second, production step (b) is operated with a limiting stream of inductive carbon-based substrate, notably of between 30 and 140 mg·g⁻¹·h⁻¹ and preferably between 35 and 45 mg·g⁻¹·h⁻¹, and preferably with an aqueous solution of inductive carbon-based substrate at a concentration of between 200 and 600 g/L.

10. The process as claimed in claim 1, characterized in that the strain used is a strain of Trichoderma reesei or of Trichoderma reesei modified by selective mutation or genetic recombination.

11. The process as claimed in claim 1, characterized in that the enzymes are cellulolytic and/or hemicellulolytic enzymes.

12. The process as claimed in claim 1, characterized in that it is operated in the absence of antifoams, in particular during the production step (a).

13. A method for the enzymatic hydrolysis of terrestrial or marine cellulosic/hemicellulosic biomass, comprising hydrolysing said biomass by the enzymes obtained by the process of claim 1.
